(19) United States
(12) Patent Application Publication
(10) Pub. No.: US 2022/0241232 A1
(43) Pub. Date: Aug. 4, 2022

(54) METHOD FOR TREATMENT OF AT RISK PATIENTS

(71) Applicant: GENFIT, LOOS (FR)

(72) Inventors: JOHN BROZEK, SAINT-AMAND-LES-EAUX (FR); NOÉMIE DAM, Vitry Sur Seine (FR); YACINE HAJJI, LILLE (FR); RÉMY HANF, LILLE (FR)

(21) Appl. No.: 17/617,011

(22) PCT Filed: Jun. 8, 2020
(86) PCT No.: PCT/EP2020/065781

§ 371 Date: Dec. 7, 2021

(30) Foreign Application Priority Data
Jun. 7, 2019 (EP) 19305742.9

(51) Int. Cl. A61K 31/201; A61K 45/06

(52) U.S. Cl. CPC A61K 31/201; A61K 45/06

(57) ABSTRACT

The invention provides an anti-fibrotic or anti-nonalcoholic steatohepatitis (NASH) drug for use in a method for reducing the risk for a subject to develop liver fibrosis or NASH, wherein the drug is administered to a subject classified as at risk to develop liver fibrosis or NASH. The invention further provides an anti-fibrotic or anti-NASH substance for use in a method for the treatment of liver fibrosis or NASH, wherein the drug is administered to a subject having type 2 diabetes.

The present invention relates to the identification of patient population at risk of developing active NASH and its consequences.

Non-alcoholic steatohepatitis (NASH) is a progressive disease of the liver characterized histologically by fatty acid accumulation, hepatocyte damage and inflammation resembling alcoholic hepatitis. NASH can lead to liver fibrosis, cirrhosis, liver failure and/or hepatocellular carninoma (HCC). Along with the obesity and type-2 diabetes rates in the world, the incidence of NASH has increased in recent years, and patients who develop NASH have an increased rate of liver-related mortalities. Since the prevalence of these diseases is increasing, the prevalence of NASH is also expected to increase and therefore, NASH has become a worldwide emerging public health issue.

The present invention relates to methods for the treatment of specific subjects at risk of NASH progression, and to drugs for use in the treatment of these subjects at risk.

In the context of the present invention, “nonalcoholic fatty liver disease”, or “NAFLD”, or “metabolic steatosis” refers to a liver condition characterized by the presence of liver steatosis in the absence of excessive alcohol consumption and after excluding other liver diseases like viral hepatitis (HCV, HBV).

According to the invention, the term “non-alcoholic steatohepatitis” refers to a NAFLD condition characterized by the concomitant presence of liver steatosis, hepatocyte ballooning and liver inflammation at histological examination, in the absence of excessive alcohol consumption and after excluding other liver diseases like viral hepatitis (HCV, HBV). According to the invention, the term “steatosis” refers to the process describing the abnormal retention of lipids or fat accumulation within the liver. According to the present invention, the term “hepatocellular ballooning” is usually defined, at the light microscopic level, based on hemotoxylin and eosin (H&E) staining, as cellular enlargement 1.5-2 times the normal hepatocyte diameter, with rarefied cytoplasm. It refers more generally to the process of hepatocyte cell death. According to the present invention, the term “lobular inflammation” refers to the presence of lobular inflammatory foci (grouped inflammatory cells) at microscopic examination of a hematoxylin and eosin (H&E) stained slice of a liver biopsy.

According to the present invention, the “NAFLD-Activity score” or “NAS” refers to the sum of steatosis, hepatocellular ballooning, lobular inflammation scores, as follows:
S: Steatosis score: 0: <5%; 1: 5-33%; 2: 34-66% and 3: >66%;
LI: Lobular Inflammation score (foci/x20 field): 0: none; 1: <2; 2: 2-4 and 3: >4;
HB: Ballooning degeneration score: 0: none; 1: few; 2: many cells/prominent ballooning.

NASH refers to a NAFLD condition characterized by the following liver biopsy-derived grades: NAS≥3, with at least 1 point in steatosis, at least 1 point in lobular inflammation and at least 1 point in the hepatocyte ballooning scores.

More severe forms of NASH are also characterized by higher grades in one of the S, LI and HB scores described above, and/or the presence of liver fibrosis. In particular, “active NASH” refers to a NASH characterized by the following liver biopsy-derived grades: NAS≥4, with at least 1 point in steatosis, at least 1 point in lobular inflammation and at least 1 point in the hepatocyte ballooning scores.

“Liver fibrosis” refers to the presence of fibrous connective tissue at microscopic examination of a stained (H&E, trichrome or picrosirius red staining) slice of a liver biopsy. In the context of the present invention, the term “fibrosis stage” denotes the localization and extent of liver fibrosis at histological exam, as follows:

| | |
|---|---|
| Perisinusoidal or periportal fibrosis | 1 |
| Mild perisinusoidal fibrosis (zone 3) | 1a |
| Moderate perisinusoidal fibrosis (zone 3) | 1b |
| Portal/periportal fibrosis | 1c |
| Perisinusoidal and portal/periportal fibrosis | 2 |
| Bridging fibrosis | 3 |
| Cirrhosis | 4 |

Alternatively, the fibrosis stage may be referred to as follows in the context of the present invention:
F=0: no fibrosis
F=1: minimal fibrosis
F=2: significant fibrosis
F=3: moderate fibrosis
F=4: severe fibrosis (i.e. cirrhosis)

According to the present invention, the term NASH refers, without limitation, to different stages of NASH, including NASH, severe NASH, active NASH, fibrosing NASH and active NASH with significant fibrosis (i.e. an active NASH characterized by liver fibrosis stage of 2 or of more than 2, such as a fibrosis stage equal to 2, 3 or 4; i.e. characterized by the following scores: NAS≥4 and F≥2). “Active NASH with significant fibrosis” can also be herein referred to by “at-risk NASH”. The method of the present invention can be used in the context of all these kinds of NASH.

According to the invention, “liver injury” denotes liver fibrosis or NASH at different stages. “Liver injury” can also refer to the consequences of NASH, such as progressive fibrosis, cirrhosis, liver failure or hepatocellular carcinoma.

In a first aspect, the invention relates to a method for reducing the risk for a subject to develop liver fibrosis or NASH, such as active NASH, more particularly active NASH with significant fibrosis, wherein the method comprises administering an anti-NASH or anti-fibrosis drug to a subject classified as at risk for liver fibrosis or for NASH, in particular for active NASH, such as for active NASH and significant fibrosis, wherein the subject is classified as at risk if the subject has type 2 diabetes. In a particular embodiment, the subject is classified as at risk if said subject has type 2 diabetes with metabolic steatosis. In a further embodiment, the classification of the subject comprises measuring the level of at least one marker selected in the group consisting of TIMP-1, CHI3L1, HA, A2M and P3NP, wherein the level of said at least one marker is compared to a predetermined threshold value, and wherein a level above or below said threshold value is indicative of a subject at risk or not at risk for liver fibrosis or NASH, such as active NASH, more particularly active NASH with significant fibrosis. In a further particular embodiment, the classification of the subject comprises the step of measuring the level of at least one of marker selected from CHI3L1 and A2M in a biological fluid sample of the subject. In yet another embodiment, the classification of the subject comprises the step of measuring the level of CHI3L1 and A2M in a biological fluid sample of the subject.

In the context of the present invention, “reducing the risk for a subject to develop liver fibrosis or NASH” should be understood to mean reducing the risk to develop liver fibrosis or NASH, but also reducing the risk of progression of liver fibrosis or reducing the risk of progression of NASH towards more advanced stages of the pathological condition.

In the context of the present invention, “TIMP-1” refers to “tissue inhibitor of metalloproteinases-1”.

In the context of the present invention, “CHI3L1” refers to “chitinase-3-like protein 1”, also known as YKL40.

In the context of the present invention, “HA” refers to “hyaluronic acid”.

In the context of the present invention, “A2M” refers to “alpha-2-macroglobulin”.

In the context of the present invention, “P3NP” refers to “procollagen type III N-terminal peptide”.

In yet another aspect, the invention relates to a method for identifying type 2 diabetes subjects in need of a treatment against liver fibrosis or NASH, in particular against active NASH, more particularly against active NASH with significant fibrosis, wherein the method comprises the step of measuring the level of at least one of the following markers in a biological fluid sample of the subject:
TIMP-1,
CHI3L-1,
HA,
A2M, and
P3NP.

In a particular embodiment, the subject has type 2 diabetes with metabolic steatosis. In a further particular embodiment, the method comprises, before the measuring step, a step of determining whether the subject has type 2 diabetes and/or metabolic steatosis. In yet another embodiment, the level of said at least one marker is compared to a predetermined threshold value, and wherein a level above or below of said threshold value is indicative of a subject in need or not in need of a treatment against liver fibrosis or NASH. In a further particular embodiment, the method comprises the step of measuring the level of at least one of marker selected from CHI3L1 and A2M in a biological fluid sample of the subject. In yet another embodiment, the method comprises the step of measuring the level of CHI3L1 and A2M in a biological fluid sample of the subject.

In a further aspect, the invention relates to a method for the identification of a subject as being at risk or not at risk of developing progressive fibrosis, cirrhosis, liver failure or hepatocellular carcinoma, said method comprising the step of determining whether the subject has type 2 diabetes. In another embodiment, the method comprises the step of determining whether the subject has type 2 diabetes and/or metabolic steatosis. In a particular embodiment, the method further comprises the step of measuring the level of at least one of the following markers in a biological fluid sample of the subject:
TIMP-1,
CHI3L-1,
HA,
A2M, and
P3NP.

In a particular embodiment, the level of said at least one marker is compared to a predetermined threshold value, and wherein a level above or below of said threshold value is indicative of a subject at risk or not at risk of developing progressive fibrosis, cirrhosis, liver failure or hepatocellular carcinoma. In a further particular embodiment, the method comprises the step of measuring the level of at least one of marker selected from CHI3L1 and A2M in a biological fluid sample of the subject. In yet another embodiment, the method comprises the step of measuring the level of CHI3L1 and A2M in a biological fluid sample of the subject.

According to yet another aspect, the invention relates to a method to identify a subject in need of clinical intervention before said subject develops liver fibrosis stage 2 or more than 2, comprising the steps of
(i) determining whether the subject has type 2 diabetes with metabolic steatosis;
(ii) measuring the level of at least one of the following markers in a biological fluid sample of the subject:
   TIMP-1,
   CHI3L1,
   HA,
   A2M, and
   P3NP.

In a particular embodiment, the level of said at least one marker is compared to a predetermined threshold value, and wherein a level above or below of said threshold value is indicative of a subject at in need or not in need of clinical intervention. In a further particular embodiment, the method comprises the step of measuring the level of at least one of marker selected from CHI3L1 and A2M in a biological fluid sample of the subject. In yet another embodiment, the method comprises the step of measuring the level of CHI3L1 and A2M in a biological fluid sample of the subject.

According to the invention, “clinical intervention” means a clinical management of the subject, for example by administering to said subject an anti-NASH and/or anti-fibrosis drug, or by providing to said subject diet and/or lifestyle advices for managing the pathological condition. In a particular embodiment of this aspect, the clinical intervention includes administration of an anti-NASH drug or an anti-fibrotic drug.

A further aspect of the invention relates to method for monitoring the efficiency of a treatment for liver fibrosis or NASH, such as for active NASH, in particular for active NASH with significant fibrosis, in a subject with type 2 diabetes.

The invention also relates, in a further aspect, to a method of treatment of liver fibrosis or NASH, in particular active NASH, more particularly active NASH with significant fibrosis, in a subject having type 2 diabetes, comprising
(i) administering an effective amount of an anti-NASH or anti-fibrosis drug to the subject,
(ii) at least one day after step (i), measuring the level of at least one marker selected from TIMP-1, CHI3L1 (YKL40), HA, A2M and P3NP in a biological fluid sample obtained from said subject,
(iii) determining whether the level of said at least one marker is above or below a predetermined threshold value; and
(iv) repeat steps (i)-(iii) until the level of said at least one marker reaches a value below said predetermined threshold value, thereby indicating that the drug has been effective to treat fibrosis or NASH, in particular active NASH, more particularly active NASH with significant fibrosis.

In a particular embodiment of this aspect, the method of treatment is a method of long term treatment.

By long term treatment, it is meant a treatment whose duration can be for more than four weeks, such as for at least one month, two months, three months, four months, five months, six months or more than six months, such as for at least one year or several years.

In a further particular embodiment, steps (ii) and (iii) are conducted at least one day after step (i), such as after 7 days after step (i), for example at least 1, 2, 3 or 4 weeks after step (i). in a particular embodiment, step (ii) and (iii) are conducted at least one month after step (i). Illustrative timings for performing steps (ii) and (iii) include, without limitation, performing steps (ii) and (iii) one month after step (i), two months after step (i) or three months after step (iii).

In a further particular embodiment, the method comprises at step (ii) the step of measuring the level of at least one of marker selected from CHI3L1 and A2M in a biological fluid sample of the subject. In yet another embodiment, the method comprises at step (ii) the step of measuring the level of CHI3L1 and A2M in a biological fluid sample of the subject.

In another aspect, the invention relates to a method for decreasing the risk of liver fibrosis and/or NASH, such as active NASH, more particularly active NASH with significant fibrosis, in a subject with type 2 diabetes, said method comprising administering to said subject an effective amount of an anti-NASH or anti-fibrosis drug. In a particular embodiment, the administration is a long term administration as defined above. In yet another embodiment, the method further comprises the step of measuring the level of at least one of the following markers in a biological fluid sample of the subject:
TIMP-1,
CHI3L-1,
HA,
A2M, and
P3NP.

In a particular embodiment, the level of said at least one marker is compared to a predetermined threshold value, and wherein a level above or below of said threshold value is indicative of a subject at risk or not at risk of developing liver fibrosis and/or NASH, such as active NASH, more particularly active NASH with significant fibrosis. In a further particular embodiment, the method comprises the step of measuring the level of at least one of marker selected from CHI3L1 and A2M in a biological fluid sample of the subject. In yet another embodiment, the method comprises the step of measuring the level of CHI3L1 and A2M in a biological fluid sample of the subject.

In yet another aspect, the invention relates to a method for reducing risks of liver fibrosis and/or NASH, such as active NASH, more particularly active NASH with significant fibrosis, associated with type 2 diabetes in a subject, wherein the method comprises administering a therapeutically effective amount of an anti-NASH or anti-fibrosis drug to said patient when the level of at least one marker selected from TIMP-1, CHI3L1, HA, A2M and P3NP measured in a biological sample from said subject is above a predetermined threshold value. In a further particular embodiment, the method comprises the step of measuring the level of at least one of marker selected from CHI3L1 and A2M in a biological fluid sample of the subject. In yet another embodiment, the method comprises the step of measuring the level of CHI3L1 and A2M in a biological fluid sample of the subject.

The invention further relates to an anti-fibrotic or anti-nonalcoholic steatohepatitis (NASH) drug for use in a method for the treatment of liver fibrosis or NASH, in particular active NASH, more particularly active NASH and significant fibrosis, in a subject in need thereof, wherein the subject to be treated has type 2 diabetes. In a particular embodiment, the subject to be treated has type 2 diabetes with metabolic steatosis. In yet a further particular embodiment, the drug is administered to a subject classified as a receiver of a treatment by measuring the level of at least one marker selected from TIMP-1, CHI3L1, HA, A2M and P3NP in a biological fluid sample of said subject, wherein the level of said at least one marker is compared to a predetermined threshold value, and wherein a level above or below of said threshold value is indicative of a subject to be classified as a receiver of a treatment. In a particular embodiment, the invention comprises measuring the level of at least one of marker selected from CHI3L1 and A2M in a biological fluid sample of the subject. In yet another embodiment, the invention comprises the step of measuring the level of CHI3L1 and A2M in a biological fluid sample of the subject.

In yet another aspect, the invention relates to an anti-fibrotic or anti-NASH drug for use in a method for the prevention of liver fibrosis or NASH, in particular of active NASH, more particularly of active NASH and significant fibrosis, in a subject in need thereof, wherein the subject to be treated has type 2 diabetes. In a particular embodiment, the subject to be treated has type 2 diabetes with metabolic steatosis. In yet a further particular embodiment, the drug is administered to a subject classified as a receiver of the drug by measuring the level of at least one marker selected from TIMP-1, CHI3L1, HA, A2M and P3NP in a biological fluid sample of said subject, wherein the level of said at least one marker is compared to a predetermined threshold value, and wherein a level above or below of said threshold value is indicative of a subject to be classified as a receiver of the drug. In a particular embodiment, the invention comprises measuring the level of at least one of marker selected from CHI3L1 and A2M in a biological fluid sample of the subject. In yet another embodiment, the invention comprises the step of measuring the level of CHI3L1 and A2M in a biological fluid sample of the subject

In a particular embodiment of the invention, the biological fluid sample can be a sample of blood, of a blood-derived fluid (for example serum or plasma, in particular platelet-free plasma, e.g. a cell-free, citrate-derived platelet-free plasma sample), of saliva, of cerebrospinal fluid or of urine. In a particular embodiment, the biological fluid is blood, plasma or serum, deprived of platelets or not. One skilled in the art will know from which body fluid a specific circulating marker should be measured. In a specific embodiment, the biological fluid sample is serum.

Illustrative anti-NASH and anti-fibrotic compounds are listed below:
a compound of formula (I):

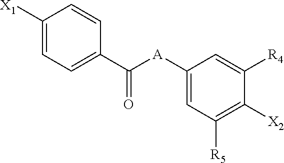

wherein:
X1 represents a halogen, a R1, or G1-R1 group;
A represents a CH═CH or a CH2-CH2 group;
X2 represents a G2-R2 group;
G1 and G2, identical or different, represent an atom of oxygen or sulfur;
R1 represents a hydrogen atom, an unsubstituted alkyl group, an aryl group or an alkyl group that is substituted by one or more halogen atoms, an alkoxy or an alkylthio group, cycloalkyl groups, cycloalkylthio groups or heterocyclic groups;
R2 represents an alkyl group substituted by at least a —COOR3 group, wherein R3 represents a hydrogen atom, or an alkyl group that is substituted or not by one or more halogen atoms, cycloalkyl groups, or heterocyclic groups.
R4 and R5, identical or different, representing an alkyl group that is substituted or not by one or more halogen atoms, cycloalkyl groups, heterocyclic groups;
or a pharmaceutically acceptable salt thereof,
Acetyl-CoA carboxylase inhibitors like GS-0976, ND-654, AC-8632, PF05175157, CP640186, gemcabene, MK-4074, and PF05175157.
Adenosine A3 receptor agonists like 2-(1-Hexynyl)-N-methyladenosine, Piclidenoson CF101 (IB-MECA), Namodenoson CF-102, 2-Cl-IB-MECA, CP-532,903, Inosine, LUF-6000, and MRS-3558.
Aldosterone antagonists and mineralocorticoid receptor antagonists like Apararenone (MT 3995), Amiloride, Spironolactone, Eplerenone, Canrenone and potassium canrenoate, progesterone, drospirenone, gestodene, and benidipine.
AMP activated protein kinase stimulators like PXL-770, MB-11055 Debio-0930B metformin, CNX-012, O-304, mangiferin calcium salt, eltrombopag, carotuximab, and Imeglimin.
Amylin receptor agonist and Calcitonin receptor agonists include, but are not limited to, KBP-042 and KBP-089.
Antisense oligonucleotide targeting transforming growth factor beta 2 include, but are not limited to ASPH-0047, IMC-TR1 and ISTH-0047.
Angiopoietin-related protein-3 inhibitors like ARO-ANG3, IONIS-ANGGPTL3-LRx or AKCEA-ANGPTL3LRx, evinacumab, and ALN-ANG.
Anti-LPS antibodies like IMM-124-E
Apical sodium-codependent bile acid transporter inhibitors like A-4250, volixibat, maralixibat formely SHP-625, GSK-2330672, elobixibat, and CJ-14199.
Betaine anhydrous or RM-003;
Bile acids like obeticholic acid (OCA) and UDCA, norursodeoxycholic acid, and ursodiol.
Bioactive lipids like 5-hydroxyeicosapentaenoic acid (15-HEPE, DS-102), unsaturated fatty acids such as 25 arachidonic acid, icosapentethyl ester, eicosapentaneoic acid, and docosahexaenoic acid.
Cannabinoid CB1 receptor antagonists like GRC-10801, MRI-1569, MRI-1867, DBPR-211, AM-6527 AM-6545, NESS-11-SM, CXB-029, GCC-2680, TM-38837, Org-50189, PF-514273, BMS-812204, ZYO-1, AZD-2207, AZD-1175, otenabant, ibipinabant, surinabant, rimonabant, drinabant, SLV-326, V-24343, and 0-2093.
Cannabinoid CB2 receptor mimetics like anabasum (Resunab, JKT-101).
Dual cannabinoid CB1 receptor/iNOS inhibitor
Caspase inhibitors like emricasan, belnacasan, nivocasan, IDN-7314, F-573, VX-166, YJP-60107, MX-1122, IDN-6734, TLC-144, SB-234470, IDN-1965, VX-799, SDZ-220-976, and L-709049.
Cathepsin inhibitors like VBY-376, VBY-825, VBY-036, VBY-129, VBY-285, Org-219517, LY3000328, RG-7236, and BF/PC-18.
CCR antagonists like cenicriviroc (CCR2/5 antagonist), PG-092, RAP-310, INCB-10820, RAP-103, PF-04634817, and CCX-872.
CCR3 chemokine modulators and eotaxin 2 ligand inhibitors.
Diacylglycerol-O-acyltransferase (DGAT) inhibitors like IONIS-DGAT2Rx formely ISIS-DGAT2Rx, LY-3202328, BH-03004, KR-69530, OT-13540, AZD-7687, ABT-046.
Dipeptidyl peptidase IV (DPP4) inhibitors like evogliptin, vidagliptin, fotagliptin, alogliptin, saxagliptin, tilogliptin, anagliptin, sitagliptin, retagliptin, melogliptin, gosogliptin, trelagliptin, teneligliptin, dutogliptin, linagliptin, gemigliptin, yogliptin, betagliptin, imigliptin, omarigliptin, vidagliptin, and denagliptin.
Insulin ligand and insulin receptor agonists.
Insulin sensitizer and MCH receptor-1 antagonis
Dual NOX (NADPH oxidase) 1&4 inhibitors like GKT-831 (2-(2-chlorophenyl)-4-[3-(dimethylamino)phenyl]-5-methyl-1H-pyrazolo[4,3-c]pyridine-3,6(2H,5H)-dione), formely GKT137831, and GKT-901.
Extracellular matrix protein modulators like CNX-024, CNX-025, and SB-030.
Stearoyl CoA desaturase-1 inhibitors/fatty acid bile acid conjugates (FABAC);
Farnesoid X receptor (FXR) agonists like obeticholic acid (OCA), GS-9674, LJN-452, EDP-305, AKN-083, INT-767, GNF-5120, LY2562175, INV-33, NTX-023-1, EP-024297, Px-103, and SR-45023.
Fatty acids like omega-3 fatty acids, Omacor or MF4637, fish oils, poly unsatured fatty acids (efamax, optiEPA).
Fatty Acid Synthase (FAS) inhibitors like TVB-2640; TVB-3199, TVB-3693BZL-101, 2-octadecynoic acid, MDX-2, Fasnall, MT-061, G28UCM, MG-28, HS-160, GSK-2194069, KD-023, and cilostazol.

In a particular embodiment, the FAS inhibitor is a compound selected in the following list of compounds:

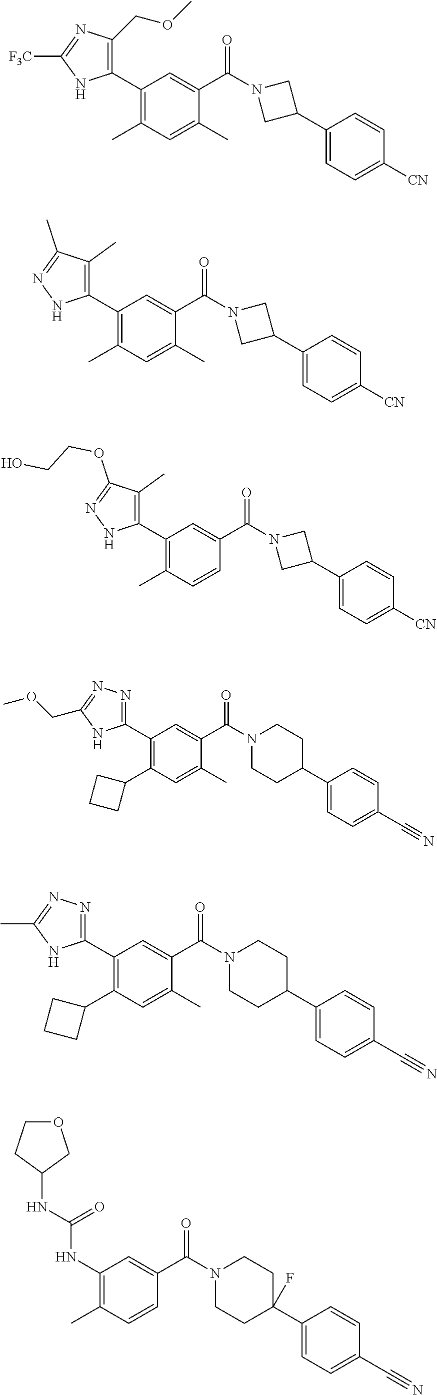 [SMILES: COCc1nc(C(F)(F)F)[nH]c1-c1cc(C(=O)N2CC(c3ccc(C#N)cc3)C2)c(C)cc1C ; Cc1cc(C)c(-c2[nH]nc(C)c2C)cc1C(=O)N1CC(c2ccc(C#N)cc2)C1 ; Cc1ccc(C(=O)N2CC(c3ccc(C#N)cc3)C2)cc1-c1[nH]nc(OCCO)c1C ; COCc1nnc(-c2cc(C(=O)N3CCC(c4ccc(C#N)cc4)CC3)c(C)cc2C2CCC2)[nH]1 ; Cc1nnc(-c2cc(C(=O)N3CCC(c4ccc(C#N)cc4)CC3)c(C)cc2C2CCC2)[nH]1 ; Cc1ccc(C(=O)N2CCC(F)(c3ccc(C#N)cc3)CC2)cc1NC(=O)NC1CCOC1]

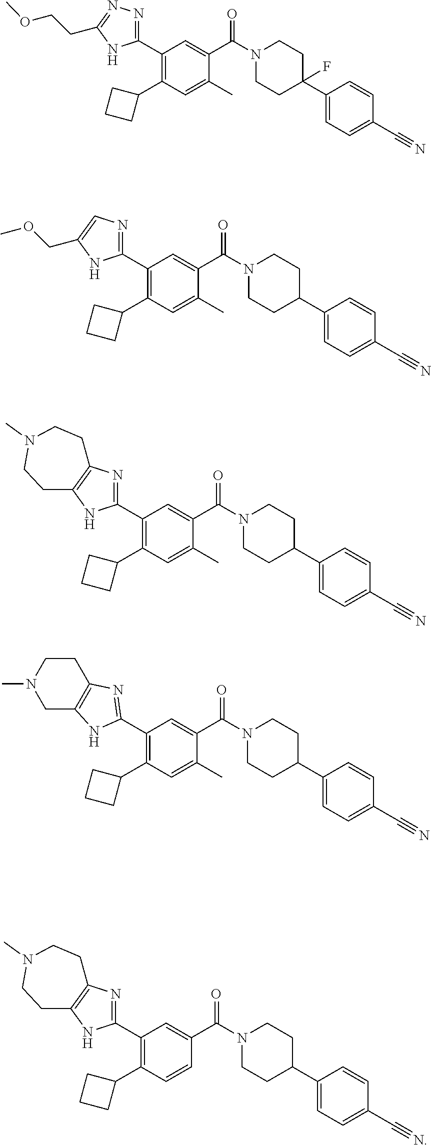 [SMILES: COCCc1nnc(-c2cc(C(=O)N3CCC(F)(c4ccc(C#N)cc4)CC3)c(C)cc2C2CCC2)[nH]1 ; COCc1cnc(-c2cc(C(=O)N3CCC(c4ccc(C#N)cc4)CC3)c(C)cc2C2CCC2)[nH]1 ; Cc1cc(C2CCC2)c(-c2nc3c([nH]2)CCN(C)CC3)cc1C(=O)N1CCC(c2ccc(C#N)cc2)CC1 ; Cc1cc(C2CCC2)c(-c2nc3c([nH]2)CN(C)CC3)cc1C(=O)N1CCC(c2ccc(C#N)cc2)CC1 ; CN1CCc2nc(-c3cc(C(=O)N4CCC(c5ccc(C#N)cc5)CC4)ccc3C3CCC3)[nH]c2CC1]

### and TVB-2640.

In another particular embodiment, the FAS inhibitor is selected from:

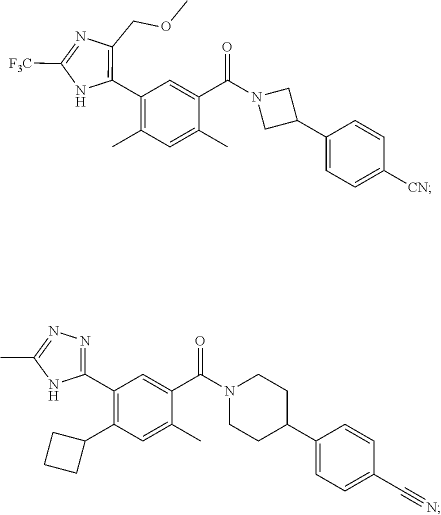 [SMILES: COCc1nc(C(F)(F)F)[nH]c1-c1cc(C(=O)N2CC(c3ccc(C#N)cc3)C2)c(C)cc1C ; Cc1nnc(-c2cc(C(=O)N3CCC(c4ccc(C#N)cc4)CC3)c(C)cc2C2CCC2)[nH]1]

### and TVB-2640.

In a particular embodiment, the FAS inhibitor is TVB-2640.
Fibroblast Growth Factor 19 (FGF-19) receptor ligand or functional engineered variant of FGF-19
Fibroblast Growth Factor 19 (FGF-19) recombinants like NGM-282
Fibroblast Growth Factor 21 (FGF-21) agonists like PEG-FGF21 formely BMS-986036, YH-25348, BMS-986171, YH-25723, LY-3025876, and NNC-0194-0499.
Galectin 3 inhibitors like GR-MD-02, TD-139, ANG-4021, Galectin-3C, LJPC-201, TFD-100, GR-MD-03, GR-MD-04, GM-MD-01, GM-CT-01, GM-CT-02, Gal-100, and Gal-200.
Glucagon-like peptide-1 (GLP-1) analogs like semaglutide, liraglutide, exenatide, albiglutide, dulaglutide, lixisenatide, loxenatide, efpeglenatide, taspoglutide, MKC-253, DLP-205, ORMD-0901.
Glucagon-like peptide-1 (GLP-1) receptor agonists like LY-3305677, and Oxyntomodulin long acting.
G-protein coupled receptor (GPCR) modulators; CNX-023.
G-protein coupled receptor 84 antagonist (GPR84 antagonist), connective tissue growth factor ligand inhibitor and Free fatty acid receptor 1 agonist (FFAR1 agonist) like PBI-4050, PBI-4265, PBI-4283, and PBI-4299.
Growth hormone
Hedgehog cell-signalling pathway inhibitors like Vismodegib, TAK-441, IPI-926, Saridegib, Sonidegib/Erismodegib, BMS-833923/XL139, PF-04449913, Taladegib/LY2940680, ETS-2400, SHR-1539, and CUR61414.
Ileal sodium bile acid cotransporter inhibitors like A-4250, GSK-2330672, volixibat, CJ-15 14199, and elobixibat.
Immunomodulators like PBI-4050, PBI-4265, PBI-4283, PBI-4299 and AIC-649.
Insulin sensitizer and MCH receptor-1 antagonist like MSDC-0602k, MSDC-0602, CSTI-100 and AMRI.
Integrin inhibitors; integrin inhibitors of Pliant Therapeutic, integrin inhibitors of Indalo Therapeutics, integrin inhibitors of St Louis University, ProAgio, and GSK-3008348.
Ketohexokinase inhibitors like JNJ-28165722, JNJ-42065426; JNJ-42152981, JNJ-42740815, JNJ-42740828, and PF-06835919.
Leukotriene (LT)/Phosphodiesterase (PDE)/Lipoxygenase (LO) inhibitors like tipelukast (formely MN-001), tomelukast, sulukast, masilukast, zafirlukast, pranlukast, montelukast, gemilukast, verlukast, aklukast, pobilikast, cinalukast, and iralukast.
Lysyl oxidase homolog 2 inhibitors like Rappaport, InterMune, Pharmaxis, AB-0023, Simtuzumab, PXS-5382A, and PXS-5338.
Macrolides: solithromycin, azithromycin, and erythromycin.
Macrophage mannose receptor modulators like AB-0023, MT-1001, [18F]FB18mHSA, Xemys, technetium Tc 99m tilmanocept, and CDX-1307.
Methyl CpG binding protein 2 modulator and transglutaminase inhibitors include, but are not limited to, cysteamine, EC Cysteamine, enteric-coated cysteamine bitartrate, cysteamine bitartrate (enteric-coated), Bennu, cysteamine bitartrate (enteric-coated), Raptor, cysteamine bitartrate, DR Cysteamine, delayed release enteric coated cysteamine bitartrate, mercaptamine, mercaptamine (enteric-coated), Bennu, mercaptamine (enteric-coated), Raptor, RP-103, RP-104, PROCYSBI, and mercaptamine (enteric-coated).
miRNA antagonists like RG-125 formely AZD4076, RGLS-5040, RG-101, MGN-5804, and MRG-201.
Metalloproteinase 9 (MMP9) stimulator like MMP9 stimulator of Elastomic Ab.
Mitochondrial carrier family inhibitor and Mitochondrial phosphate carrier protein inhibitor include, but are not limited to TRO-19622, Trophos, olesoxime, RG-6083, or RO-7090919.
Myeloperoxidase inhibitors include, but are not limited to PF-06667272
Monoclonal antibodies: bertilimumab, NGM-313, IL-20 targeting mAbs, fresolimumab (antiTGFβ) formely GC1008, timolumab formely BTT-1023, namacizumab, omalizumab, ranibizumab, bevacizumab, lebrikizumab, epratuzumab, felvizumab, matuzumab, monalizumab, reslizumab, and inebilizumab.
Monoclonal antibodies like anti-IL20 mAbs, anti-TGFβ antibodies, anti-CD3 antibodies, anti-LOXL2 antibodies and anti-TNF antibodies.
mTOR modulators like MSDC-0602, AAV gene therapy co-administered with SVP-sirolimus.
NAD-dependent deacetylase sirtuin stimulator, PDE 5 inhibitor like NS-0200.
NF-kappa B inhibitors like LC-280126.
niclosamide and its derivatives,
Nicotinic acid like Niacin or Vitamine B3
Nicotinic Acid Receptor (GPR109) Agonists like ARI-3037MO, MMF, LUF 6283, Acifran, IBC 293, MK-1903, GSK256073, MK-6892, MK-0354, SLx-4090, lomitapide, lexibulin, apabetalone, acifran, laropiprant, daporinad, anacetrapib, INCB-19602, ST-07-02, lomefloxacin, Niacin, and controlled release/laropiprant,
nitazoxanide (NTZ), its active metabolite tizoxanide (TZ) or other prodrugs of TZ such as RM-5061,
non-steroid anti-inflammatory drugs (NSAIDs) include, but are not limited to F-351, salicylates (aspirin), acetaminophen, propionic acid derivatives (ibuprofen, naproxen), acetic acid derivatives (indomethacin, diclofenac), enolic acid derivatives (piroxicam, phenylbutazone), anthranilic acid derivatives (meclofenalmic acid, flufenamic acid), selective 25 COX-2 inhibitors (celecoxib, parecoxib), and sulfonanilides (nimesulide).
nuclear receptor ligands like DUR-928 formely DV 928.
P2Y 13 protein agonists like CER-209
PDGFR modulators like BOT-501 and BOT-191.
Phenylalanine hydroxylase stimulators like Pegvaliase, sapropterin, AAV-PAH, CDX-6114, sepiapterin, RMN-168, ALTU-236, ETX-101, HepaStem, rolipram, and alprostadil
Protease-activated receptor (PAR)-2 antagonists; PZ-235, and NP-003.
Protein kinase modulators like CNX-014, MB-11055, ALF-1, mangiferin, amlexanox, GS-444217, REG-101, and valine.
PPAR alpha agonists like fenofibrate, ciprofibrate, pemafibrate, gemfibrozil, clofibrate, binifibrate, clinofibrate, clofibric acid, nicofibrate, pirifibrate, plafibride, ronifibrate, theofibrate, tocofibrate, and SR10171;
PPAR gamma agonists like Pioglitazone, deuterated pioglitazone, Rosiglitazone, efatutazone, ATx08-001, OMS-405, CHS-131, THR-0921, SER-150-DN, KDT-501, GED-0507-34-Levo, CLC-3001, and ALL-4.
PPAR delta agonists like GW501516 (Endurabol or ({4-[({4-methyl-2-[4-(trifluoromethyl)phenyl]-1,3-thiazol-5-yl}methyl)sulfanyl]-2-methylphenoxy}acetic acid)) or MBX8025 (Seladelpar or {2-methyl-4-[5-methyl-2-(4-trifluoromethyl-phenyl)-2H-[1,2,3]triazol-4-ylmethylsylfanyl]-phenoxy}-acetic acid) or GW0742 ([4-[[[2-[3-fluoro-4-(trifluoromethyl)phenyl]-4-methyl-5-thiazolyl]methyl]thio]-2-methyl phenoxy]acetic acid) or L165041 or HPP-593 or NCP-1046.
PPAR alpha/gamma agonists (also named glitazars), like Saroglitazar, Aleglitazar, Muraglitazar, Tesaglitazar, and DSP-8658.
PPAR alpha/delta agonists like Elafibranor, and T913659.
PPAR gamma/delta like conjugated linoleic acid (CLA), T3D-959.
PPAR alpha/gamma/delta agonists or PPARpan agonists: IVA337 or TTA (tetradecylthioacetic acid) or Bavachinin or GW4148 or GW9135, or Bezafibrate or Lobeglitazone, or CS038.
Prebiotic fibers, probiotics
Pregnane X receptors like Rifampicin.
Rho-associated protein kinase 2 (ROCK2) inhibitors: KD-025, TRX-101, BA-1049, LYC-53976, INS-117548, and RKI-1447.
signal-regulating kinase 1 (ASK1) inhibitors; GS-4997
Sodium-glucose transport (SGLT) 2 inhibitors: remogliflozin, dapagliflozin, empagliflozin, ertugliflozin, sotagliflozin, ipragliflozin, tianagliflozin, canagliflozin, tofogliflozin, janagliflozin, bexagliflozin, luseogliflozin, sergliflozin, HEC-44616, AST-1935, and PLD-101.
stearoyl CoA desaturase-1 inhibitors/fatty acid bile acid conjugates: aramchol, GRC-9332, steamchol, TSN-2998, GSK-1940029, and XEN-801.
thyroid receptor β (THR β) agonists: VK-2809, MGL-3196, MGL-3745, SKL-14763, sobetirome, BCT-304, ZYT-1, MB-07811, and eprotirome.
Toll Like Receptor 4 (TLR-4) antagonists like naltrexone, JKB-121, M-62812, resatorvid, dendrophilin, CS-4771, AyuV-1, AyuV-25, NI-0101, EDA-HPVE7, and eritoran.
Tyrosine kinase receptor (RTK) modulators; CNX-025; KBP-7018
Urate anion exchanger 1 inhibitors and xanthine oxidase inhibitors like lesinurad, RLBN-1001, verinurad, KUX-1151, and lesinurad+allopurinol.
Vascular adhesion protein-1 (VAP-1) inhibitors like PXS-4728A, CP-664511, PRX-167700, ASP-8232, RTU-1096, RTU-007, and BTT-1023.
Vitamin D receptor (VDR) agonists like calciferol, alfacalcidol, 1,25-dihydroxyvitamin D3, Vitamin D2, Vitamin D3, calcitriol, Vitamin D4, Vitamin D5, dihydrotachysterol, calcipotriol; tacalcitol 1,24-dihydroxyvitamin D3, and paricalcitol.
Vitamin E and isoforms, vitamin E combined with vitamin C and atorvastatin;
and pharmaceutically acceptable salts of these drugs.

Other anti-NASH agents include KB-GE-001 and NGM-386 and NGM-395 and NC-10 and TCM-606F. Further anti-NASH agents include icosabutate, NC-101, NAIA-101 colesevelam, and PRC-4016. Other anti-fibrotic agents include HEC-585, INV-240, RNAi therapeutic (Silence Therapeutics) and SAMiRNA program (Bioneer Corp). Other illustrative antifibrotic agents include pirfenidone or receptor tyrosine kinase inhibitors (RTKIs) such as Nintedanib, Sorafenib and other RTKIs, or angiotensin II (AT1) receptor blockers, or CTGF inhibitor, or any antifibrotic compound susceptible to interfere with the TGFβ and BMP-activated pathways including activators of the latent TGFβ complex such as MMP2, MMP9, THBS1 or cell-surface integrins, TGFβ receptors type I (TGFBRI) or type II (TGFBRII) and their ligands such as TGFβ, Activin, inhibin, Nodal, anti-Müllerian hormone, GDFs or BMPs, auxiliary co-receptors (also known as type III receptors), or components of the SMAD-dependent canonical pathway including regulatory or inhibitory SMAD proteins, or members of the SMAD-independent or non-canonical pathways including various branches of MAPK signaling, TAK1, Rho-like GTPase signaling pathways, phosphatidylinositol-3 kinase/AKT pathways, TGFβ-induced EMT process, or canonical and non-canonical Hedgehog signaling pathways including Hh ligands or target genes, or any members of the WNT, or Notch pathways which are susceptible to influence TGFβ.

In a particular embodiment the treatment of NASH, or active NASH, or active NASH with significant fibrosis or liver fibrosis comprises administering a compound of formula (I) selected in the group consisting of 1-[4-methylthiophenyl]-3-[3,5-dimethyl-4-carboxydimethylmethyloxy phenyl]prop-2-en-1-one, 1-[4-methylthiophenyl]-3-[3,5-dimethyl-4-isopropyloxy carbonyldimethylmethyloxyphenyl]prop-2-en-1-one, 1-[4-methylthiophenyl]-3-[3,5-dimethyl-4-tertbutyloxycarbonyldimethylmethyloxyphenyl] prop-2-en-1-one, 1-[4-trifluoromethylphenyl]-3-[3,5-dimethyl-4-tertbutyloxycarbonyl dimethylmethyloxyphenyl]prop-2-en-1-one, 1-[4-trifluoromethylphenyl]-3-[3,5-dimethyl-4-carboxydimethylmethyloxyphenyl]prop-2-en-1-one, 1-[4-trifluoromethyl oxyphenyl]-3-[3,5-dimethyl-4-tertbutyloxycarbonyldimethylmethyloxy phenyl] prop-2-en-1-one, 1-[4-trifluoromethyloxyphenyl]-3-[3,5-dimethyl-4-carboxydimethylmethyl oxyphenyl]prop-2-en-1-one, 2-[2,6-dimethyl-4-[3-[4-(methylthio)phenyl]-3-oxo-propyl] phenoxy]-2-methylpropanoic acid, and 2-[2,6-dimethyl-4-[3-[4-(methylthio) phenyl]-3-oxo-propyl]phenoxy]-2-methyl-propanoic acid isopropyl ester; or a pharmaceutically acceptable salt thereof. In a further particular embodiment of the invention, the compound of formula (I) is 1-[4-methylthiophenyl]-3-[3,5-dimethyl-4-carboxydimethylmethyloxy phenyl]prop-2-en-1-one or a pharmaceutically acceptable salt thereof.

In particular, the invention relates to a combination product comprising at least an anti-NASH and/or an anti-fibrotic agent for use in a method for treating NASH, in particular active NASH, more particularly active NASH with significant fibrosis, in a subject in need thereof, wherein the subject has been classified as a receiver of said treatment thanks to a method according to the invention.

In a more particular embodiment, the invention relates to the treatment of NASH, in particular active NASH, more particularly active NASH with significant fibrosis, with a combination product comprising at least one agent selected from the group of anti-NASH and/or anti-fibrotic compounds, or pharmaceutically acceptable salts thereof.

In a more particular embodiment, the invention relates to the treatment of NASH, in particular active NASH, more particularly NASH with significant fibrosis with Elafibranor or a pharmaceutically acceptable salt thereof.

In a further embodiment the treatment of NASH, in particular active NASH, more particularly active NASH with significant fibrosis, comprises administering NTZ, TZ, vitamin E, pioglitazone, obeticholic acid, elafibranor, selonsertib, saroglitazar, semaglutide, niclosamide, bezafibrate and/or cenicrivoc, or a pharmaceutically acceptable salt thereof.

In a further embodiment, the treatment of NASH, in particular active NASH, more particularly active NASH with significant fibrosis, comprises administering NTZ or TZ, or a pharmaceutically acceptable salt thereof in particular NTZ or a pharmaceutically acceptable salt thereof.

In a further particular embodiment, a combination treatment is conducted. In another particular embodiment, the treatment of NASH, in particular active NASH, more particularly active NASH with significant fibrosis comprises administering Elafibranor combined with one or more other NASH or anti-liver fibrosis compound. In yet another embodiment, the treatment of NASH, in particular active NASH, more particularly active NASH with significant fibrosis, comprises administering Elafibranor combined with at least one compound selected in the group consisting of NTZ, TZ, vitamin E, pioglitazone, obeticholic acid, semaglutide, niclosamide, selonsertib, saroglitazar, bezafibrate and cenicrivoc, or a pharmaceutically acceptable salt thereof. In yet another embodiment, the treatment of NASH, in particular active NASH with significant fibrosis comprises administering Elafibranor, or a pharmaceutically acceptable salt thereof combined with NTZ or a pharmaceutically acceptable salt thereof.

The drugs used in the invention can be formulated into pharmaceutical compositions. The pharmaceutical compositions used in the invention can comprise one or several excipients or vehicles, acceptable within a pharmaceutical context (e.g. saline solutions, physiological solutions, isotonic solutions, etc., compatible with pharmaceutical usage and well-known by one of ordinary skill in the art). These compositions can also comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. The drugs can be formulated for enteral or parenteral administration. For example, the drugs can be formulated for oral, intravascular (e.g. intravenous or intra-arterial), intramuscular, intraperitoneal, subcutaneous, transdermal or nasal administration. The formulation can be a solid or liquid dosage form. Illustrative formulations include, without limitation, an injectable suspension, or suspension for oral ingestion, a gel, an oil, a pill, a tablet, a suppository, a powder, a capsule, an aerosol, an ointment, a cream, a patch, or means of galenic forms for a prolonged and/or slow release. For this kind of formulation, agents such as cellulose, carbonates or starches can be advantageously used.

The drugs implemented herein can be formulated as pharmaceutically acceptable salts, particularly acid or base salts compatible with pharmaceutical use. Salts of the compounds implemented herein include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound.

In a particular embodiment, the active drugs can be for administration as one or more pharmaceutical composition(s) in the form of a pill or tablet intended for an oral ingestion.

In another embodiment, the drugs are for administration as one or more pharmaceutical composition(s) in the form of injectable solutions.

In a further particular embodiment, for treatments with drug combinations, the drugs are for administration as separate compositions.

The frequency of administration and/or dose of the drug can be adapted by one of ordinary skill in the art, in function of the subject to be treated, the disease to be treated, the stage of the disease, the form of administration, etc. Illustratively, a drug can be administered, without limitation, at a dose comprised between 0.01 mg/day to 4000 mg/day. For example, elafibranor or a pharmaceutically acceptable salt thereof can be administered at a dose comprised between 0.01 mg/day to 4000 mg/day, such as from 1 mg/day to 2000 mg/day, in particular from 25 to 1000 mg/day, particularly from 50 to 200 mg/day, and even more particularly from 80 to 120 mg/day.

Administration can be performed daily or even several times per day, if necessary. The duration of the treatment will depend on the specific disease to be treated. For example, the administration can be performed during one or several days, such as during at least one day, at least two days, at least three days, at least four days, at least five days, at six two days or at least seven days. Alternatively, the administration can be performed for at least one week, at least two weeks, at least four weeks. In a particular embodiment, the treatment is a long term treatment, with an administration that can be considered for more than four weeks, such as for at least one month, two months, three months, four months, five months, six months or more than six months, such as for at least one year or several years.

In some cases, the combination product of the invention can be administered during the lifetime of the subject.

The invention is further described with reference to the following, non-limiting, examples.

### EXAMPLES

### Description of the Figures

FIG. 1: Prevalence of T2D according to liver status.

FIG. 2: Type 2 Diabetes as a risk factor for «at-risk» NASH.

FIG. 3: Type 2 Diabetes as risk factor for active NASH.

FIG. 4: Type 2 Diabetes as a risk factor for liver fibrosis.

### MATERIALS AND METHODS

RESOLVE-IT is a Multicenter, Randomized, Double-Blind, Placebo-Controlled Phase III Study (NCT02704403) to Evaluate the Efficacy and Safety of Elafibranor in Patients with Nonalcoholic Steatohepatitis (NASH) and fibrosis. The study was approved by appropriate regulatory bodies all patients had given informed consent for participation. An inclusion liver biopsy was used for examination and scoring of histological lesions. Blood samples were withdrawn at screening. In patients who have signed a dedicated informed consent, additional blood samples were collected for research of new diagnostic biomarkers of NASH. 2363 patients with metabolic steatosis were screened for inclusion in the phase 3 elafibranor trial RESOLVE-IT with a liver biopsy centrally scored for NAS and F-stage.

This study includes patients “at risk” or to be treated (NAS≥4 and F≥2) and not to be treated patients (NAS<4 and F<2).

Metabolic syndrome is defined by central obesity (waist circumference >=94 cm in men and >=80 cm in women) and at least 2 of the following factors: serum triglycerides >=1.70 mmol/L or specific treatment for this lipid abnormality; serum high-density lipoprotein (HDL) cholesterol <1.03 mmol/L in men and <1.29 mmol/L in women or specific treatment for this lipid abnormality; systolic blood pressure (BP) >=130 mm Hg or diastolic BP >=85 mm Hg or treatment for previously diagnosed hypertension; fasting plasma glucose >=5.6 mmol/L or previously diagnosed type 2 diabetes.

Prevalence of active disease (NAS≥4), significant fibrosis (F≥2) and progressive NASH (NAS≥4+F≥2, i.e. “at-risk” of hepatic outcomes) and clinical and biochemical NASH markers were assessed according to the T2D status.

Blood was collected in SST tube. In order to ensure a good barrier between cells and serum, the SST tubes have to be centrifuged for 10 minutes at room temperature between 1300-2000 g. Serum was then transferred in a new vial and frozen at −70° C. The EDTA tube(s) 3 mL for Hematology/HbA1c were kept at room temperature until analysis. Blood was collected in fluoride tube to measure fasting plasma glucose.

To identify hypertension and dyslipidemia, co-treatments variable were used.

Results

Characteristics of Patients

2363 patients with metabolic steatosis were screened for inclusion in the phase 3 elafibranor trial RESOLVE-IT (NTC02704403) with a liver biopsy centrally scored for NAS and F-stage. 71% of patients of the cohort were obese, 47% suffered from dyslipidemia, 81% Metabolic syndrome. 35% of the cohort had T2D, 88% of them were managed by >1 oral antidiabetic or insulin.

TABLE 1
Characteristics of Patients with Type 2 Diabetes and
non diabetic patients.

| | Non-T2D | Type 2 Diabetics | |
|---|---|---|---|
| | (N = 1528) | (N = 835) | P values |
| Age (year) | 51.5 (±12.4) | 56.6 (±10.1) | P < 0.001 |
| Gender (M/F) | 65%/35% | 54%/46% | P < 0.001 |
| Weight (kg) | 96.1 ± 20.7 | 96.9 ± 21.0 | NS |
| BMI (kg/m2) | 33 ± 6 | 34 ± 6 | P < 0.001 |
| T2D (%) | 0 | 100 | — |
| Dyslipidemia (%) | 39% | 63% | P < 0.001 |
| Hypertension (%) | 48% | 73% | P < 0.001 |
| Metabolic Syndrome (%) | 73% | 95% | P < 0.001 |
| Fasting Plasma glucose | 5.40 ± 1.24 | 7.143 ± 2.24 | P < 0.001 |
| (mmol/L) | | | |
| Fasting Insulin (pmol/L) | 193 ± 217 | 235 ± 344 | P < 0.001 |
| HOMA-IR | 6.9 ± 10.6 | 11.4 ± 24.5 | P < 0.001 |
| HbA1C (%) | 5.74 ± 0.72 | 6.97 ± 1.08 | P < 0.001 |
| Triglycerides (mmol/L) | 1.97 ± 1.12 | 2.16 ± 1.38 | P < 0.001 |
| Total-Cholesterol (mmol/L) | 5.00 ± 1.17 | 4.55 ± 1.19 | P < 0.001 |
| LDL-Cholesterol (mmol/L) | 2.90 ± 0.94 | 2.39 ± 0.96 | P < 0.001 |
| HDL-Cholesterol (mmol/L) | 1.22 ± 0.38 | 1.19 ± 0.31 | NS |
| ALT (U/L) | 64.6 ± 46.6 | 60.1 ± 44.3 | P = 0.008 |
| AST (U/L) | 44.2 ± 30.3 | 44.8 ± 31.6 | NS |
| GGT (U/L) | 74.9 ± 96.4 | 79.0 ± 84.8 | P < 0.001 |
| ALP (U/L) | 83.7 ± 32.9 | 84.3 ± 33.2 | NS |
| CK18-M30 (U/L) | 618 ± 577 | 705 ± 614 | P < 0.001 |
| CK18-M65 (U/L) | 583 ± 645 | 607 ± 598 | NS |
| TIMP1 (ng/ml) | 254 ± 70 | 268 ± 69 | P < 0.001 |
| P3NP (ng/ml) | 10.7 ± 5.5 | 10.9 ± 5.7 | NS |
| Hyaluronic Acid (ng/ml) | 64.1 ± 84.4 | 82.6 ± 104.7 | P < 0.001 |
| Alpha2-macroglobulin (g/IL) | 2.27 ± 0.90 | 2.51 ± 0.90 | P < 0.001 |

Mean ± SD, p-values were determined by Pearson's Chi Squared and Wilcoxon tests for categorical and continuous data, respectively.

Prevalence of Type 2 Diabetes and at-Risk NASH

Prevalence of active disease (NAS≥4), significant fibrosis (F≥2) and progressive NASH (NAS≥4+F≥2, i.e. “at-risk” of hepatic outcomes) and clinical and biochemical NASH markers were assessed according to the T2D status.

The prevalence of T2D is significantly higher in patients at risk of fibrosis progression defined by the presence of NAS≥4 and F≥2 (OR=2.20, 95% CI [1.85; 2.62]; p<0.001) (FIG. 1). The prevalence of “at-risk” NASH was 65% in T2D (n=835), and 45% for non-T2Ds (n=1528).

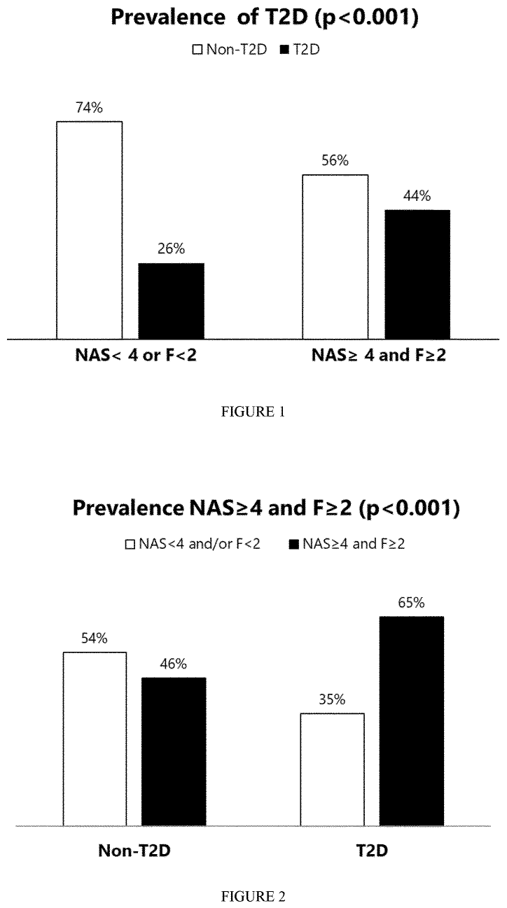

T2D was a significant risk factor (p<0.001) for “at-risk” NASH (OR=2.20, 95% CI [1.85; 2.62]) (FIG. 2).

The association between T2D and “at risk” NASH was independent of age, gender or BMI.

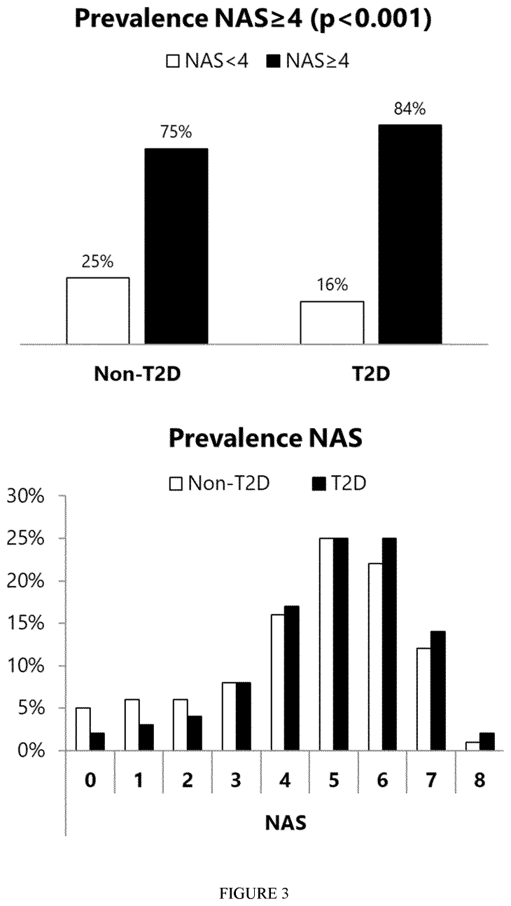

Significant difference in the prevalence of active NASH (NAS≥4) was observed in T2D vs Non-T2D patients (FIG. 3).

T2D patients had higher mean NAS scores (4.96 vs. non-T2Ds: 4.47; p<0.001).

T2D is a significant risk factor (p<0.001) for NAS≥4 (OR=1.74, 95% CI [1.40;2.16]).

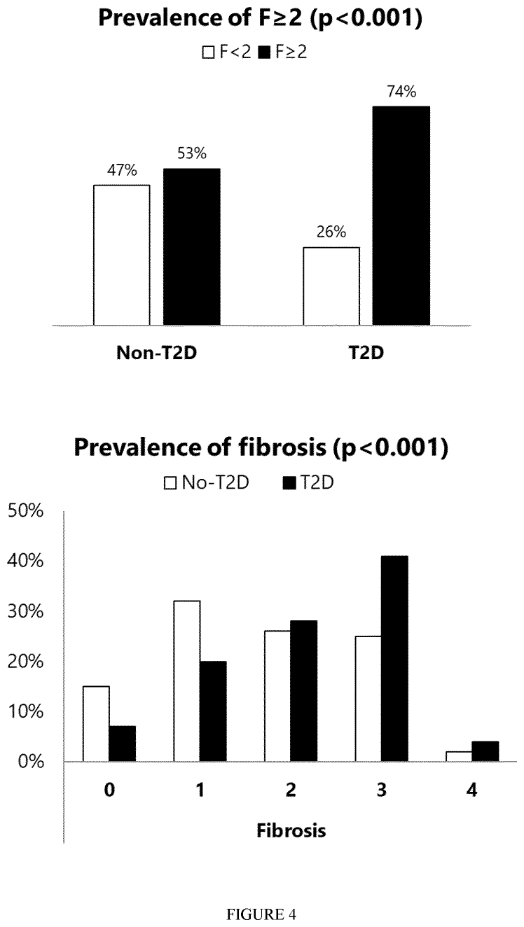

The prevalence of significant (F≥2) and advanced fibrosis (F3-4) is significantly higher in T2D patients (FIG. 4).

T2D patients had higher mean fibrosis stage (2.17 vs. non-T2D: 1.67; p<0.001).

T2D is a significant risk factor (p<0.001) for F≥2 (OR=2.50, 95% CI [2.08;3.01]), although 94% of patients were treated with anti-diabetic drugs. 45% of T2D patients have advanced fibrosis, ie are at high risk of cirrhosis and liver failure.

In both non-T2D and T2D, HbA1c and glucose homeostasis markers remained higher in “at-risk” NASH (p<0.001). Histological lesions and fibrosis are associated with markers of insulin resistance. Almost all T2D patients are treated by OADs and/or insulin, but HbA1c and other markers of glucose homeostasis remain significantly elevated vs non-diabetic patients.

Liver injury markers ALT, AST, GGT or CK18 were higher (p<0.001) in “at-risk” NASH patients but were similar in T2Ds vs. non-T2Ds. Plasma lipids are not associated with the presence or absence of NASH and fibrosis. Well controlled lipid profiles in T2D are probably caused by a higher % of lipid-lowering treatment in these patients.

Fibrosis serum markers such as TIMP-1, CHI3L1, HA, A2M, or P3NP were elevated in “at-risk” NASH patients (p<0.001) in T2D and non T2D patients.

TABLE 2
Association of individual parameters with NASH at risk state and T2D.

| Parameter | | NAS < 4 or F < 2 | NAS ≥ 4 and F ≥ 2 | P |
|---|---|---|---|---|
| FPG (mmol/L) | Non-T2D | 5.22 ± 1.10 | 5.63 ± 1.35 | <0.001 |
| | T2D | 6.75 ± 2.01 | 7.33 ± 2.32 | <0.001 |
| | P | <0.001 | <0.001 | |
| Insulin (pmol/L) | Non-T2D | 157 ± 143 | 234 ± 273 | 0.03 |
| | T2D | 199 ± 235 | 254 ± 388 | <0.001 |
| | P | <0.001 | NS | |
| HOMA-IR | Non-T2D | 5.4 ± 6.4 | 8.68 ± 13.9 | <0.001 |
| | T2D | 9.0 ± 12.5 | 12.8 ± 28.9 | 0.04 |
| | P | <0.001 | 0.001 | |
| HBA1c (%) | Non-T2D | 5.63 ± 0.65 | 5.86 ± 0.78 | <0.001 |
| | T2D | 6.77 ± 1.06 | 7.07 ± 1.08 | <0.001 |
| | P | <0.001 | <0.001 | |
| Fructosamine (umol/L) | Non-T2D | 230 ± 28 | 242 ± 35 | <0.001 |
| | T2D | 259 ± 43 | 273 ± 49 | 0.03 |
| | P | <0.001 | <0.001 | |
| ALT (U/L) | Non-T2D | 53 ± 37 | 78 ± 53 | <0.001 |
| | T2D | 45 ± 32 | 68 ± 48 | <0.001 |
| | P | 0.002 | <0.001 | |
| AST (U/L) | Non-T2D | 35 ± 20 | 55 ± 36 | <0.001 |
| | T2D | 32 ± 21 | 52 ± 34 | <0.001 |
| | P | 0.04 | NS | |
| GGT (U/L) | Non-T2D | 64 ± 77 | 88 ± 113 | <0.001 |
| | T2D | 64 ± 80 | 87 ± 86 | <0.001 |
| | P | NS | NS | |
| ALP (U/L) | Non-T2D | 82 ± 32 | 86 ± 33 | 0.014 |
| | T2D | 84 ± 32 | 84 ± 34 | NS |
| | P | NS | NS | |
| CK18-M30 (U/L) | Non-T2D | 443 ± 357 | 789 ± 691 | <0.001 |
| | T2D | 474 ± 383 | 820 ± 672 | <0.001 |
| | P | NS | NS | |
| CK18-M65 (U/L) | Non-T2D | 442 ± 420 | 649 ± 717 | <0.001 |
| | T2D | 473 ± 407 | 644 ± 637 | 0.003 |
| | P | NS | NS | |
| TIMP1 (ng/ml) | Non-T2D | 234 ± 51 | 273 ± 81 | <0.001 |
| | T2D | 240 ± 52 | 282 ± 72 | <0.001 |
| | P | NS | NS | |
| CHI3L1 (pg/ml) | Non-T2D | 56 ± 53 | 100 ± 126 | <0.001 |
| | T2D | 70 ± 61 | 115 ± 114 | <0.001 |
| | P | <0.001 | 0.04 | |
| Hyaluronic acid (ng/ml) | Non-T2D | 48 ± 70 | 80 ± 94 | <0.001 |
| | T2D | 57 ± 50 | 96 ± 122 | <0.001 |
| | P | NS | 0.014 | |
| A2M (g/L) | Non-T2D | 2.03 ± 0.83 | 2.50 ± 0.90 | <0.001 |
| | T2D | 2.27 ± 0.86 | 2.63 ± 0.89 | <0.001 |
| | P | <0.001 | 0.015 | |
| P3NP (ng/ml) | Non-T2D | 8.9 ± 4.0 | 12.4 ± 6.2 | <0.001 |
| | T2D | 8.5 ± 3.2 | 12.1 ± 6.3 | <0.001 |
| | P | NS | NS | |
| TG (mmol/L) | Non-T2D | 1.97 ± 1.20 | 1.96 ± 1.04 | NS |
| | T2D | 2.06 ± 1.45 | 2.21 ± 1.33 | NS |
| | P | NS | <0.001 | |
| VLDL-Chol (mmol/L) | Non-T2D | 0.89 ± 0.34 | 0.85 ± 0.38 | NS |
| | T2D | 0.94 ± 0.36 | 0.92 ± 0.40 | NS |
| | P | NS | 0.007 | |
| Total Chol (mmol/L) | Non-T2D | 5.02 ± 1.19 | 4.99 ± 1.15 | NS |
| | T2D | 4.49 ± 1.16 | 4.58 ± 1.20 | NS |
| | P | <0.001 | <0.001 | |
| LDL-Chol (mmol/L) | Non-T2D | 3.04 ± 0.92 | 2.87 ± 0.94 | NS |
| | T2D | 2.50 ± 0.87 | 2.37 ± 0.97 | NS |
| | P | <0.001 | <0.001 | |
| HDL-Chol (mmol/L) | Non-T2D | 1.26 ± 0.36 | 1.21 ± 0.38 | NS |
| | T2D | 1.23 ± 0.38 | 1.19 ± 0.30 | NS |
| | P | NS | NS | |

In conclusion, in patients with metabolic steatosis, T2D is associated with necroinflammation and fibrosis, and independently increases the risk of “at-risk” NASH (NAS≥4 and F≥2).

In contrast to liver enzymes, fibrosis markers are significantly higher in T2D patients compared to non-T2D regardless of histological category; this is in concordance with the observed higher degree of fibrosis of T2D patients.

These data accentuate the need for active surveillance of liver injury in T2D patients, in order to identify those in need of clinical intervention before they develop clinically relevant hepatic fibrosis.

TIMP1, CHI3L1, Hyaluronic acid, P3NP and A2M parameters were higher in “at risk” NASH patients (p<0.001) versus non at risk patients in T2Ds and non-T2D patients.

CHI3L1 and A2M parameters were also significantly higher in T2Ds versus non-T2D patients in “non at risk” and “at risk” subgroups.

1-11. (canceled)

12. A method for reducing the risk of developing liver fibrosis or nonalcoholic steatohepatitis (NASH) in a subject, wherein an anti-fibrotic or anti-NASH drug is administered to a subject classified as at risk to develop liver fibrosis or NASH and the subject is classified as at risk if said subject has type 2 diabetes.

13. The method according to claim 12, wherein the subject has type 2 diabetes with metabolic steatosis.

14. The method according to claim 12, wherein the subject is further identified by measuring the level, in a biological fluid sample of the subject, of at least one marker selected from the group consisting of:
TIMP-1,
CHI3L1 (YKL-40),
HA,
A2M, and
P3NP.

15. The method according to claim 14, wherein the at least one marker is selected from CHI3L1 and A2M.

16. The method according to claim 15, wherein the levels of CHI3L1 and A2M are measured.

17. The method according to claim 12, wherein the subject has liver fibrosis and the liver fibrosis stage 1, 2, 3 or 4.

18. The method according to claim 12, wherein the subject has active NASH or active NASH with significant fibrosis.

19. The method according to claim 12, wherein the drug is a PPAR agonist, a FXR agonist, a CCR antagonist or a ASK1 inhibitor.

20. The method according to claim 19, wherein the drug is selected from the group consisting of elafibranor, NTZ, TZ, vitamin E, pioglitazone, obeticholic acid, selonsertib, saroglitazar, niclosamide, bezafibrate, cenicrivoc, and pharmaceutically acceptable salts thereof.

21. A method of treating liver fibrosis or nonalcoholic steatohepatitis (NASH) comprising the administration of an anti-fibrotic or anti-NASH drug to a subject having type 2 diabetes.

22. The method according to claim 21, wherein the subject has type 2 diabetes with metabolic steatosis.

23. The method according to claim 21, wherein the method further comprises measuring the level, in a biological fluid sample of the subject, of at least one marker selected from the group consisting of:
TIMP-1,
CHI3L1 (YKL-40),
HA,
A2M, and
P3NP.

24. The method according to claim 21, wherein the subject has type 2 diabetes and the method comprises the steps of:
(i) administering an anti-NASH or anti-fibrosis drug to the subject,
(ii) at least one day after step (i), measuring the level of at least one marker selected from TIMP-1, CHI3L1 (YKL40), HA, A2M and P3NP in a biological fluid sample obtained from said subject,
(iii) determining whether the level of said at least one marker is above or below a predetermined threshold value; and
(iv) repeat steps (i)-(iii) until the level of said at least one marker reaches a value below said predetermined threshold value, thereby indicating that the drug has been effective in treating fibrosis or NASH.

25. The method according to claim 23, wherein the at least one marker is selected from CHI3L1 and A2M.

26. The method according to claim 25, wherein the levels of CHI3L1 and A2M are measured.

27. The method according to claim 21, wherein the subject has liver fibrosis and the liver fibrosis is stage 1, 2, 3 or 4.

28. The method according to claim 21, wherein NASH is active NASH or active NASH with significant fibrosis.

29. The method according to claim 21, wherein the drug is a PPAR agonist, a FXR agonist, a CCR antagonist or a ASK1 inhibitor.

30. The method according to claim 29, wherein the drug is selected in the group consisting of elafibranor, NTZ, TZ, vitamin E, pioglitazone, obeticholic acid, selonsertib, saroglitazar, niclosamide, bezafibrate, cenicrivoc, and pharmaceutically acceptable salts thereof.
